(19) United States
(12) Patent Application Publication
(10) Pub. No.: US 2026/0185961 A1
(43) Pub. Date: Jul. 2, 2026

(54) CLAY VOLUME IDENTIFICATION

(71) Applicant: Schlumberger Technology Corporation, Sugar Land, TX (US)

(72) Inventors: Azizah Al Radhwan, Dhahran (SA); Ahmad Mohamad AlZoukani, Dhahran (SA); Mohammed Fadhel Al-Hamad, Dhahran (SA); Farhan Ali, Dhahran (SA); Olivier Sindt, Dhahran (SA)

(21) Appl. No.: 19/422,677

(22) Filed: Dec. 17, 2025

Related U.S. Application Data
(60) Provisional application No. 63/739,736, filed on Dec. 30, 2024.

(51) Int. Cl. G01N 27/447

(52) U.S. Cl. CPC G01N 27/447

(57) ABSTRACT

A method to quantity clay content may comprise flowing a rock sample with water, wherein the water is collected, and a capillary electrophoresis analysis or any water analysis method is run on the water. The method further comprises calculating a clay integrated area using constructed water analysis peaks. Lastly, a correlation to quantify the clay content in the rock sample, and another correlation to quantify the clay content in the water sample are formulated.

### BACKGROUND

Many sandstone reservoirs contain clay minerals like kaolinite, illite, and chlorite in abundance. Quantification of clay minerals is crucial for the assessment of clay-rich rock and soil; however, it is difficult because of their distinct structures and varied elemental compositions. There are different methods which can be used to analyze minerals including X-ray diffraction (XRD), Fourier transform infrared spectroscopy (FTIR), chemical analysis and electron microscopy. Among these methods, XRD is one of the most important analytical techniques for qualitative and quantitative analysis of the geological samples. Nevertheless, the quantitative analysis of particular minerals, especially clay, still remains a challenge because of the diverse chemical compositions, preferred orientations, structural disorder, and vast structural variety of clay minerals (Srodon, 2002).

Clay, particularly, illite is highly sensitive to low pH fluids and fresh water. Water is adsorbed in the interlayer spaces of some clay minerals and interferes with clay quantitative analysis. Furthermore, when saturated with water, montmorillonite contains two planes of water molecules in the interlayer space. As a result, the interlayer water molecules significantly alter the XRD reflections of clay minerals, preventing a quantitative analysis of the XRD spectra. In addition, the precision of quantitative analysis is highly reliant on the sample preparation methods like the fine grinding of the samples and XRD instrument conditions.

### BRIEF DESCRIPTION OF THE DRAWINGS

The present disclosure is best understood from the following detailed description when read with the accompanying Figures. It is emphasized that, in accordance with the standard practice in the industry, various features are not drawn to scale. In fact, the dimensions of the various features may be arbitrarily increased or reduced for clarity of discussion.

FIG. 1 is a summarized workflow to calculate the clay content using CE analysis, according to one or more examples of the disclosure.

FIG. 2 is an electropherogram of 1% illite in water, according to one or more examples of the disclosure.

FIG. 3 is an electropherogram of 2% illite in water, according to one or more examples of the disclosure.

FIG. 4 is an electropherogram of 4% illite in water according to one or more examples of the disclosure.

FIG. 5 is an electropherogram of 8% illite in water, according to one or more examples of the disclosure.

FIG. 6 is a series electropherograms of the 1%-8% water samples from 2 min until 4 min., according to one or more examples of the disclosure.

FIG. 7 is a graph of illite content [%] versus total area of illite [%], according to one or more examples of the disclosure.

FIG. 8 is a graph of illite content versus the calculated clay content using the developed correlation, according to one or more examples of the disclosure.

### DETAILED DESCRIPTION

Illustrative examples of the subject matter claimed below will now be disclosed. In the interest of clarity, not all features of an actual implementation are described in this specification. It will be appreciated that in the development of any such actual implementation, numerous implementation-specific decisions may be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

Further, as used herein, the article “a” is intended to have its ordinary meaning in the patent arts, namely “one or more.” Herein, the term “about” when applied to a value generally means within the tolerance range of the equipment used to produce the value, or in some examples, means plus or minus 10%, or plus or minus 5%, or plus or minus 1%, unless otherwise expressly specified. Further, herein the term “substantially” as used herein means a majority, or almost all, or all, or an amount with a range of about 51% to about 100%, for example. Moreover, examples herein are intended to be illustrative only and are presented for discussion purposes and not by way of limitation.

The present invention discloses a technique to quantify clay minerals in rock samples from water analysis using capillary electrophoresis (CE). This method aimed to develop a reliable CE method using UV detection for the quantification of clay from water analysis. CE is a technique that separates compounds based on their charge. Moreover, the present technique is also a cost-effective method that quantifies the clay in the rock samples in a single run using CE coupled with a UV detector in 7 minutes only.

### Methodology and Results

Different weight ratios of clay in water (1%, 2%, 4%, and 8%) were used. Illite clay was used as an example of clay mineral in the graphs below. The Chemical structure of illite is K_{1-1.5}Al₄(Si_{7-8.5},Al_{1-1.5})O₂₀(OH)₄·, which is mainly composed of alumina, silica, and water, with other associated elements like potassium, magnesium, and iron.

The illite samples were left in water for one day and then were removed. The water samples were analysed using CE cation analysis. FIG. 1 illustrates the summarized workflow of the present method to calculate clay content using CE analysis, which is further described below.

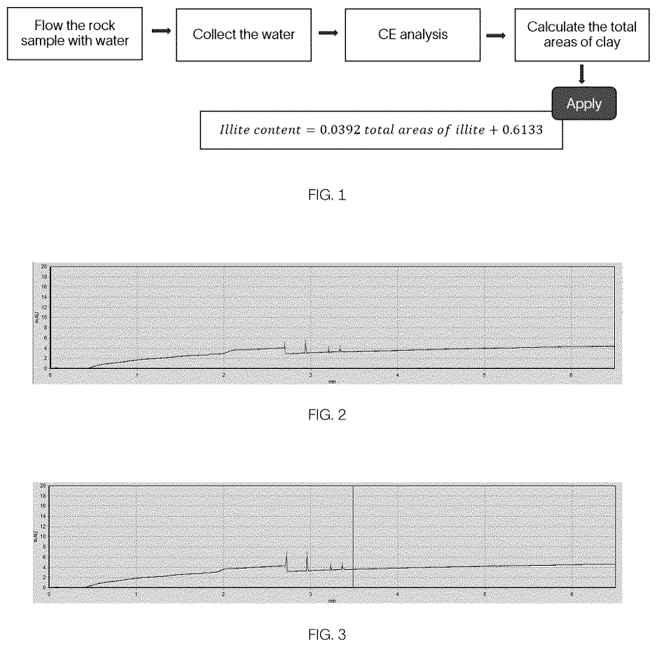

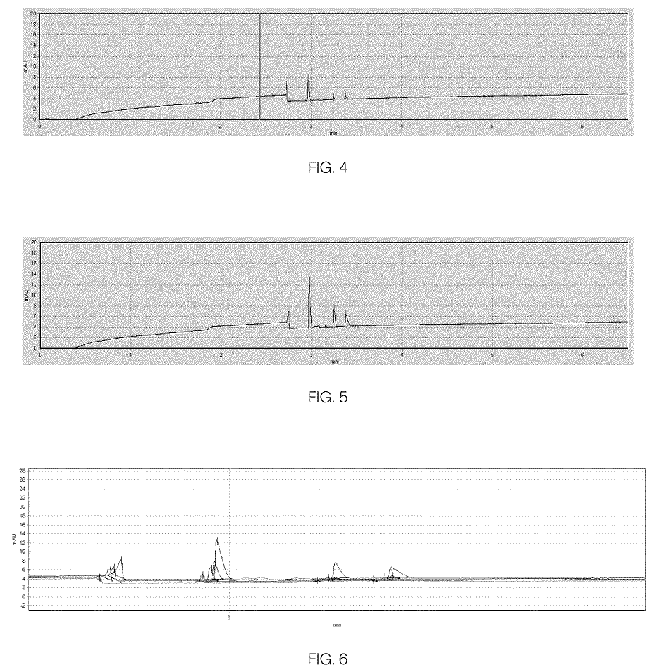

FIGS. 2 to 5 illustrates the electropherograms of the water solutions after the removal of the illite samples (1%-8% illite in water). It is observed that the electropherograms exhibited similar migration times. However, the area under the peaks which indicate the concentration of the ions where highly different. The higher the area under the peaks, the higher the concentration of the ions which represent the concentration of clay in the samples.

From the above test, it is revealed that the area under the peaks located between two and four minutes were significantly increased by increasing the concentration of clay, which is future illustrated in FIG. 6. At the same time, Table 1 below summarizes the total calculated area for the four different concentrations (1%-8%).

TABLE 1
The total calculated area for the four samples
(1%-8%) concentration of illite in water.

| Area for 1% | Area for 2% | Area for 4% | Area for 8% |
|---|---|---|---|
| Illite in Water | Illite in Water | Illite in Water | Illite in Water |
| solution | solution | solution | solution |
| 19.403 | 49.932 | 58.664 | 192.34 |

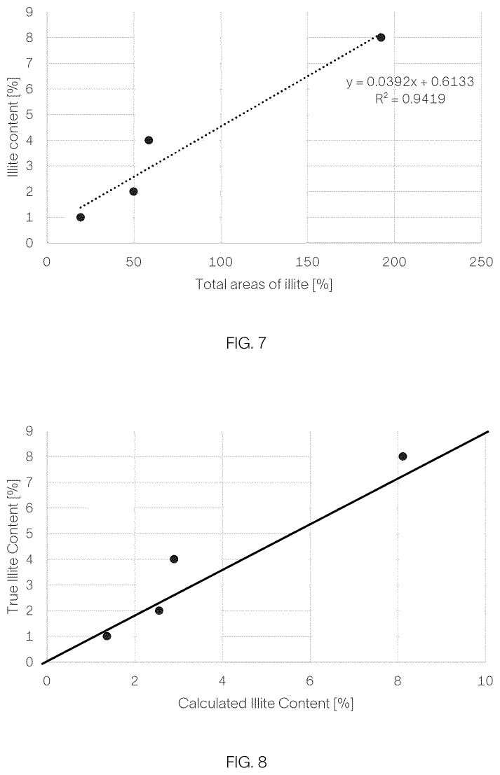

The data from Table 1 shows a robust correlation between the clay concentration and the total areas of illite from the CE electropherograms. The data were plotted as shown in FIG. 7, resulting in a good correlation coefficient of R²=0.9419, and the fitting equation between the illite content and the total areas of illite is shown in equation (1):

$$Illite content = 0.0392 total areas of illite + 0.6133 \qquad ( 1 )$$

Using the fitting equation (1) will help to calculate the illite content form the rock samples through water analysis measurements.

### Estimation of the Accuracy of the Methodology

Using equation (1) the illite content of the four samples were calculated and the results summarized in Table 2. The error calculations are shown in the Table 2 and the plotted data in FIG. 8.

TABLE 2
Calculated illite content using the developed equation

| | Clay Content | Calculated Clay | Absolute |
|---|---|---|---|
| Sample Name | [%] | Content [%] | Error |
| 1% Illite/Water | 1 | 1.37 | 0.37 |
| 2% Illite/Water | 2 | 2.57 | 0.56 |
| 4% Illite/Water | 4 | 2.91 | 1.09 |
| 8% Illite/Water | 8 | 8.15 | 0.11 |

The correlation developed in this method provides a good estimate of the illite content when compared to the actual clay content present in the samples with maximum average absolute error of 0.77.

Also, the uniqueness of this technique is that it is sensitive to small amounts of clay present in the sample, as highlighted by the 1% illite/water which would not be measured using the conventional methods.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Obviously, many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

What is claimed is:

1. A method to quantify clay content by flowing rock sample with clay with water, wherein the water is collected and a capillary electrophoresis analysis is run on the water.

2. The method of claim 1, wherein the water is collected, and any water analysis method is run on the water.

3. The method of claim 1, wherein a clay integrated area of the clay is calculated using constructed water analysis peaks.

4. The method of claim 1, wherein a correlation to quantify the clay content in the rock sample is formulated.

5. The method of claim 1, wherein a correlation to quantify the clay content in the water sample is formulated.
